(19) United States
(12) Patent Application Publication
(10) Pub. No.: US 2013/0289347 A1
(43) Pub. Date: Oct. 31, 2013

(54) ENDOSCOPIC SYSTEM

(71) Applicant: OLYMPUS CORPORATION, Tokyo (JP)

(72) Inventors: Takeshi ITO, Hino-shi (JP); Masahiro NISHIO, Hachioji-shi (JP); Eiji YAMAMOTO, Musashimurayama-shi (JP)

(21) Appl. No.: 13/932,330

(22) Filed: Jul. 1, 2013

Related U.S. Application Data
(63) Continuation of application No. PCT/JP2012/050370, filed on Jan. 11, 2012.

(30) Foreign Application Priority Data
Jan. 12, 2011 (JP) 2011-004118

(51) Int. Cl. A61B 1/06

(52) U.S. Cl. CPC A61B 1/06
USPC 600/102; 600/103

(57) ABSTRACT

An endoscopic system is a system which an insertion portion of an endoscope is inserted from an insertion opening of an object to observe an inner surface of the object. The endoscopic system includes an electromagnetic radiation unit configured to radiate electromagnetic waves, a detection section configured to detect the electromagnetic waves, and a determination section configured to determine whether the insertion portion is present in the object based on a detection result of the detection section. One of the electromagnetic radiation unit and the detection section is arranged outside the object, and the other is arranged at the insertion portion.

### CROSS REFERENCE TO RELATED APPLICATIONS

This application is a Continuation Application of PCT Application No. PCT/JP2012/050370, filed Jan. 11, 2012 and based upon and claiming the benefit of priority from prior Japanese Patent Application No. 2011-004118, filed Jan. 12, 2011, the entire contents of all of which are incorporated herein by reference.

### BACKGROUND OF THE INVENTION

1. Field of the Invention

The present invention relates to an endoscopic system which an insertion portion of an endoscope is inserted from an insertion opening of an object to observe an inner surface of the object.

2. Description of the Related Art

In an endoscope, as a light source configured to illuminate an inner surface of an object which is an observation target, a light source having a small luminous point such as a laser or a light source that radiates light having relatively high energy like ultraviolet light or blue light is used.

With respect to radiated light radiated from such a light source apparatus, a maximum permissible exposure (MPE) of a human body largely varies depending on eyes and skin. That is, the MPE for the skin has a value which is several score times larger than the MPE for eyes. Thus, in a biological endoscope, there has been desired detection means for detecting whether an insertion portion of an endoscope having an illumination light exit portion arranged at a distal end thereof. When such detection means is provided, a light volume upper limit based the MPE for eyes is set for the outside of a body, a light volume upper limit for skin is set for the inside of a body, and control can be effected so that the light source can emit light with a light volume required for observation.

Further, for the purpose of preventing a subject from feeling annoyed with glare, detection means for detecting that an insertion portion is present inside or outside a body is desired.

On the other hand, in an industrial endoscope, to extend life duration of a light source apparatus or achieve power saving, when the insertion portion is present outside an observation target object, detection means for detecting the inside or the outside of the observation target object is likewise desired for the purpose of stopping or dimming the light source.

In contrast, Japanese Patent No. 4316118 discloses a technology that detects the inside of a living body or the outside of a living body by detecting flicker of a fluorescent lamp by means of a detector disposed at a distal end of a scope.

However, the technology disclosed in Japanese Patent No. 4316118 uses the flicker of the fluorescent lamp. Therefore, in the biological endoscope cannot detect that the insertion portion is present in the inside or the outside of a body in an examination room where the fluorescent lamp is not used. On the other hand, in the industrial endoscope, whether the insertion portion is present in the inside or the outside of the observation target object cannot be detected in an outdoor usage environment. Further, even in a case where a fluorescent lamp is provided in a room, if any other illumination apparatus is also provided or if intensive external light enters from a window or the like, flicker of the fluorescent light is masked by such light and may not be detected with certainty.

In view of the above-described point, it is an object of the present invention to provide an endoscopic system that makes it possible to detect with certainty that an insertion portion is present inside or outside an object under any illumination conditions.

### BRIEF SUMMARY OF THE INVENTION

According to a first aspect of the invention, there is provided an endoscopic system which an insertion portion of an endoscope is inserted from an insertion opening of an object to observe an inner surface of the object, comprising:

an electromagnetic radiation unit configured to radiate electromagnetic waves;

a detection section configured to detect the electromagnetic waves; and

a determination section configured to determine whether the insertion portion is present in the object based on a detection result of the detection section,

wherein one of the electromagnetic radiation unit and the detection section is arranged outside the object, and the other is arranged at the insertion portion.

According to the present invention, it is possible to provide an endoscopic system in which an electromagnetic radiation unit actively radiates electromagnetic waves and whether an insertion portion of an endoscope is present in an object is determined based on a detection state of the electromagnetic waves in the insertion portion, and hence whether the insertion portion is present in the object or outside the object can be detected with certainty under any illumination conditions.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic block diagram of an endoscopic system according to a first embodiment of the present invention;

FIG. 2 is a view showing an operation flowchart of the endoscopic system according to the first embodiment;

FIG. 3 is a view showing a configuration of the endoscopic system according to the first embodiment;

FIG. 4 is a view showing a configuration concerning illumination of the endoscopic system according to the first embodiment;

FIG. 5 is a perspective view showing a scope distal end portion in the endoscopic system according to the first embodiment;

FIG. 6 is a view showing a configuration of an electromagnetic radiation unit in the endoscopic system according to the first embodiment;

FIG. 7A is a cross-sectional view showing a configuration of an electromagnetic wave detector in an endoscopic system according to a second embodiment of the present invention;

FIG. 7B is a perspective view showing the configuration of the electromagnetic wave detector in the endoscopic system according to the second embodiment;

FIG. 8 is a perspective view showing another structural example of the electromagnetic wave detector;

FIG. 9 is a perspective view showing an operating portion and its vicinity in a scope section in an endoscopic system according to a third embodiment of the present invention;

FIG. 10 is a perspective view showing an operating portion and its vicinity in a scope section in a modification of the endoscopic system according to the third embodiment;

FIG. 11 is a view showing an arrangement position of an electromagnetic radiation unit in an endoscopic system according to a fourth embodiment of the present invention;

FIG. 12 is a perspective view showing a configuration of an insertion portion in an endoscopic system according to a fifth embodiment of the present invention; and

FIG. 13 is a schematic block diagram of an endoscopic system according to a seventh embodiment of the present invention.

### DETAILED DESCRIPTION OF THE INVENTION

Embodiments of the present invention will now be described with reference to the drawings.

### First Embodiment

As shown in FIG. 1, an endoscopic system according to a first embodiment of the present invention is constituted of an insertion portion 10, an electromagnetic radiation unit 12, an electromagnetic wave detector 14, and a determination section 16. The insertion portion 10 is an insertion portion of an endoscope that is inserted from an insertion opening I of an object O. The electromagnetic radiation unit 12 radiates electromagnetic waves. The electromagnetic wave detector 14 is a detection section that detects the electromagnetic waves radiated from the electromagnetic radiation unit 12. The determination section 16 determines whether the insertion portion 10 is present in the inside of object O_{I} based on a detection result of the electromagnetic wave detector 14. Here, the electromagnetic radiation unit 12 is arranged in the outside of object O_{O}, and the electromagnetic wave detector 14 is arranged at the insertion portion 10.

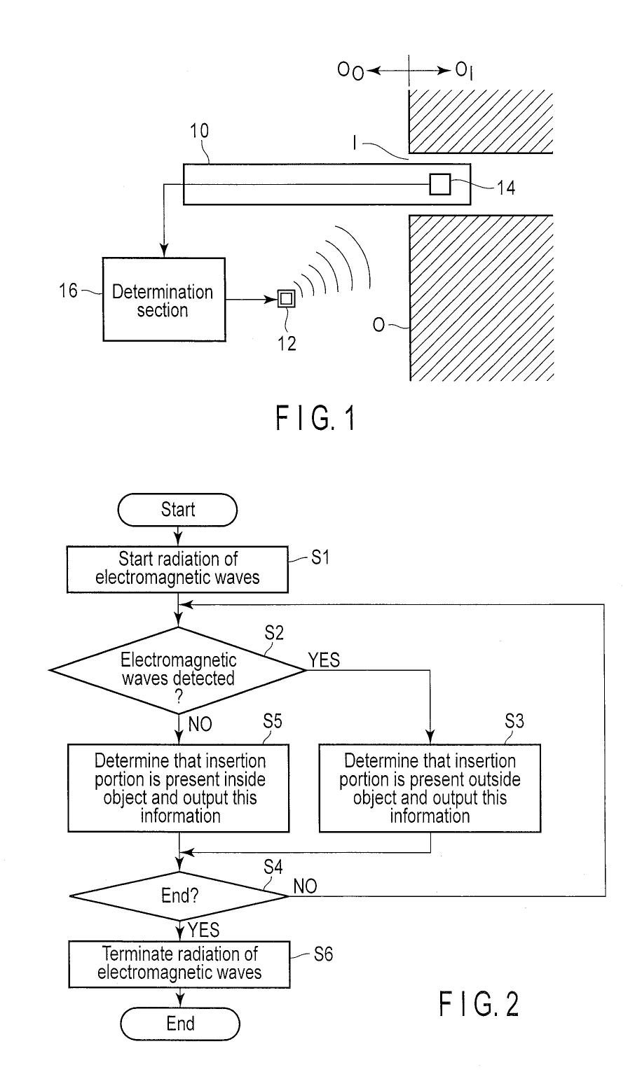

In such an endoscopic system, as shown in FIG. 2, the determination section 16 first allows the electromagnetic radiation unit 12 to radiate the electromagnetic waves with start of an operation of the endoscope (step S1).

Then, the determination section 16 receives a detection result from the electromagnetic detector 14 and determines whether the electromagnetic waves have been detected by the electromagnetic wave detector 14 from this detection result (step S2). Here, if the determination section 16 has been determined that the electromagnetic waves were detected by the electromagnetic detector 14, it determines that the insertion portion 10 is present in the outside of object O_{O}, and outputs information indicative of this determination (step S3). Thereafter, the determination section 16 determines whether an operation of the endoscope has been terminated (step S4), and returns to step S2 if the operation is not completed.

On the other hand, if the determination section 16 has been determined that the electromagnetic waves were no longer detected by the electromagnetic wave detector 14 in step S2, it determines that the insertion portion 10 is present in the inside of object O_{I}, and outputs information indicative of this determination (step S5). Subsequently, the processing of the determination section 16 advances to step S4, the section 16 determines whether the operation of the endoscope is completed, and returns to step S2 if the operation is not completed.

Furthermore, if the operation of the endoscope was determined to be completed in step S4, the determination section 16 allows the electromagnetic radiation unit 12 to finish radiation of the electromagnetic waves (step S6) and terminates the operation.

It is to be noted that any determination result is output here but, but if at least one of the determination results is output, a member that receives outputs from the determination section 16 can recognize in which one of the inside of object O_{I} or the outside of object O_{O} the insertion portion 10 is present based on whether the determination result has been output.

A more specific configuration will now be described hereinafter based on an example where the endoscope is a biological endoscope.

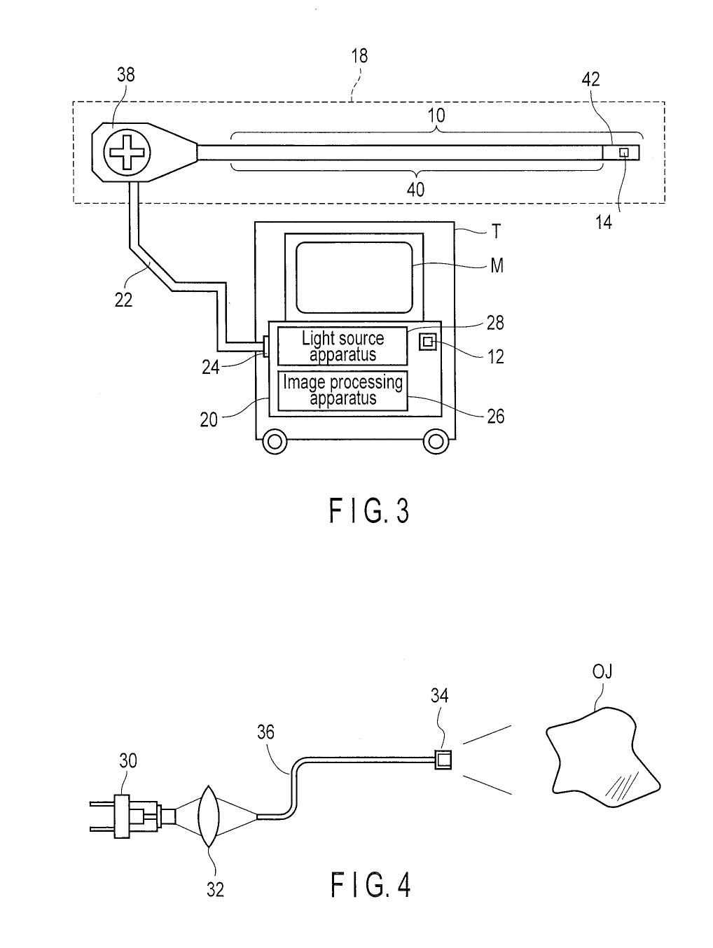

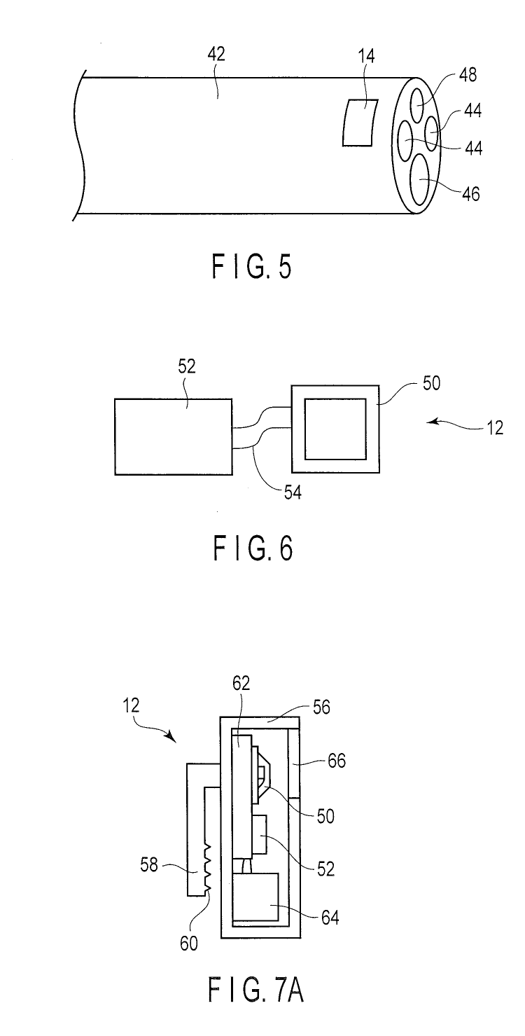

As shown in FIG. 3, the biological endoscope can be divided into a scope section 18 which is held by an operator such as a physician to perform an operation and a main body section 20 mounted in a trolley T. Specifically, a connection cable 22 extending from the scope section 18 is attachable to or detachable from a connecting portion 24, for example, a connector or the like in the main body section 20.

It is to be noted that the trolley T means a movable rack in which the endoscope is mounted, and a monitor M, a printer that prints acquired images, and others as well as the main body section 20 are mounted. Although not shown in particular, a holding portion that holds the scope section 18 is provided in this trolley T so that the scope section 18 can be suspended and held in a state that the scope section 18 is connected to the main body section 20. At the time of use, the scope section 18 can be removed from the holding portion and then used.

The main body section 20 includes an image processing apparatus (a video processor) 26 and various other members required for endoscopic observation. The image processing apparatus 26 supplies electric power to the scope section 18 or processes images acquired by an imaging section (which will be described later) arranged at a distal end of the scope section 18. As other members, for example, a light source apparatus 28 configured to radiate illumination light from the distal end of the scope section 18 is included. The main body section 20 is connected to the monitor M which displays, for example, images acquired by the imaging section.

It is to be noted that the determination section 16 may be configured in the image processing apparatus 26, may be configured in the light source apparatus 28, or may be configured in the main body section 20 to be independent from these members.

Furthermore, the above-described electromagnetic radiation unit 12 is arranged at the main body section 20. It is to be noted that FIG. 3 shows an example where the image processing apparatus 26 and the light source apparatus 28 are incorporated in one housing of the main body section 20. However, the apparatuses may be combined by using different housings so that one main body section 20 can be configured. In the latter case, the electromagnetic radiation unit 12 may be incorporated in either the housing of the image processing apparatus 26 or that of the light source apparatus 28.

For example, as shown in FIG. 4, the light source apparatus 28 includes an excitation light source 30, an optical system 32, and a light source control section (not shown). The excitation light source 30 is a laser with a small luminous point or an LED that emits light that has relatively high energy such as ultraviolet light or blue light. The optical system 32 condenses excitation light from the excitation light source 30. The light source control section controls an amount of luminescence or light emission timing of the excitation light source 30. On the other hand, an illumination object OJ must be illuminated with light having a wavelength suitable for observation, for example, white light. Thus, a wavelength converting section 34 is mounted at a distal end portion of the scope section 18. Furthermore, the excitation light source 30 and the wavelength converting section 34 are connected through an optical fiber 36. That is, the optical fiber 36 is arranged in the connection cable 22 and the scope section 18. Therefore, when the wavelength converting section 34 is irradiated with the excitation light emitted from the excitation light source 30 through the optical fiber 36, the wavelength converting section 34 radiates the illumination light, and the illumination light is applied to the illumination object OJ.

In addition, it is needless to say an electric wiring line as well as the optical fiber 36 is formed in the connection cable 22 between the scope section 18 and the main body section 20.

On the other hand, the scope section 18 is formed of the insertion portion 10 and the operating portion 38. The insertion portion 10 is operated to be inserted into a lumen of a living body when an operator, for example, a physician holds a portion near a distal end of the insertion portion 10 with his/her right hand. The operating portion 38 is held with the operator's left hand and operated. The insertion portion 10 is formed of a bending portion 40 and a hard portion 42. The bending portion 40 is configured to be readily deformed in accordance with bend of a lumen, and it bends in response to an operation of the operating portion 38. The hard portion 42 is provided at a distal end portion of the bending portion 40, and it does not deform. Two illumination light exit portions 44 from which the illumination light exits, an imaging section 46 which acquires images, and a channel 48 into which a forceps or the like is inserted are provided on a distal end surface of this hard portion 42. The wavelength converting section 34 is mounted in the hard portion 42 for the illumination light exit portions 44. Furthermore, a non-illustrated imaging element such as a CCD is mounted in the hard portion 42 for the imaging section 46.

Moreover, the above-described electromagnetic wave detector 14 is provided on a side surface of the hard portion 42 of the insertion portion 10. Non-illustrated wiring lines, for example, a power supply wiring line through which electric power is supplied, a signal wiring line through which a detected signal is transmitted, and others extends in the insertion portion 10 from this electromagnetic wave detector 14 toward the operating portion 38.

It is to be noted that, in regard to an installing position of the electromagnetic wave detector 14, at the time of inserting the insertion portion 10 into a lumen of a subject, there is an appropriate depth at which determining that the insertion portion 10 is present in the inside of object O_{I}, i.e., the inside of a body is desirable when inserted to a given depth. For example, in case of inserting the insertion portion 10 from, for example, a mouth to an esophagus and a stomach which are lumens, the subject's throat region must be brightly illuminated in order to insert the insertion portion 10 with certainty into the esophagus. Therefore, considering a length from a lip region to the throat region which is an oral length, it is specifically desirable for the electromagnetic wave detector 14 to be installed in the range of approximately 5 cm from an end portion of the distal end of the hard portion 42. Additionally, to avoid a situation where the inside of a body is detected at the moment that the hard portion 42 is inserted into the mouth, it is desirable to arrange the electromagnetic wave detector 14 at a position which is 1 cm or more apart from the end portion.

Further, when image noise may be possibly produced when the imaging section 46 in the hard portion 42 is affected by electromagnetic waves, it is desirable to shield the entire hard portion 42 with a member through which electromagnetic waves hardly pass. At this moment, the electromagnetic wave detector 14 must be arranged outside the shield, or an opening must be formed in the shield and the electromagnetic wave detector 14 must be arranged in this opening.

The electromagnetic waves radiated from the electromagnetic radiation unit 12 and detected by the electromagnetic wave detector 14 must be, for example, infrared rays in a wavelength region that is not transmitted through a human body or electric waves in a wavelength region that is sufficiently attenuated by a human tissue which is several centimeter thick. A description will be given on the assumption that the electromagnetic radiation unit 12 is an infrared radiation element and the electromagnetic wave detector 14 is an infrared ray detector. However, it is needless to say that the electromagnetic radiation unit 12 may be formed of a radio wave radiation element and the electromagnetic wave detector 14 may be formed of a radio wave detector.

As shown in FIG. 6, the electromagnetic radiation unit 12 is constituted of an infrared LED 50 that radiates infrared rays and an electromagnetic wave control circuit 52. The infrared LED 50 and the electromagnetic wave control circuit 52 are connected through wiring lines 54. The electromagnetic wave control circuit 52 has a function of controlling a radiation amount, a modulation pattern, and others of the electromagnetic waves radiated from the infrared LED 50. Here, this circuit is configured to modulate, for example, blink the electromagnetic waves radiated from the infrared LED 50 in a predetermined pattern so that electromagnetic waves having the same wavelength radiated from a different device cannot be erroneously detected. Therefore, the electromagnetic wave control circuit 52 has a non-illustrated memory section that stores the predetermined modulation pattern (a blinking pattern). The electromagnetic wave control circuit 52 supplies electric power to the infrared LED 50 through the wiring lines 54 in a predetermined blinking pattern previously stored in this memory section, thereby blinking and emitting light.

It is to be noted that the electromagnetic wave control circuit 52 is connected to a non-illustrated power supply in the main body section 20 through a non-illustrated electric wiring line.

As described above, the endoscopic system, using a biological endoscope has a configuration that the electromagnetic wave detector 14 provided in the hard portion 42 at the distal end of the insertion portion 10 of the scope section 18 detects the electromagnetic waves (infrared rays) radiated from the electromagnetic radiation unit 12. Since the infrared rays hardly pass through a human body, when the hard portion 42 at the distal end of the insertion portion 10 is inserted into the lumen, for example, the inside of a mouth, the electromagnetic wave detector 14 that detects infrared rays hardly detect infrared rays radiated from the electromagnetic radiation unit 12. On the other hand, since the infrared rays are efficiently propagated through space, they are detected with certainty by the electromagnetic wave detector 14 if there is no obstruction.

The electromagnetic radiation unit 12 is previously installed at the trolley T or the main body section 20 mounted thereon. Since the main body section 20 on the trolley T is connected to the scope section 18, a positional relationship between the trolley T, the main body section 20, the scope section 18, a subject, and an operator (a physician) is substantially determined. Therefore, the electromagnetic radiation unit 12 is previously installed at such a position on the trolley T or the main body section 20 as that the insertion opening I of the lumen of the subject can be irradiated with certainty with the infrared rays. As a result, the insertion opening I of the lumen can be irradiated with certainty with the infrared rays. Consequently, it is possible to substantially eliminate a situation that the hard portion 42 at the distal end of the scope section 18 is hidden behind an operator or any other member in a room, and the electromagnetic wave detector 14 cannot detect the infrared rays and the determination section 16 determines that the hard portion 42 is present inside a body even though the hard portion 42 at the distal end of the scope section 18 is present outside the body. Further, in this embodiment, light from an interior lamp or the like is not used, the dedicated emitter (the electromagnetic radiation unit 12) and receiver (the electromagnetic wave detector 14) are used. Furthermore, the electromagnetic radiation unit 12 blinks in a predetermined blinking pattern. Therefore, the determination section 16 can detect with certainty whether the electromagnetic wave detector 14, i.e., the insertion portion 10 is present in the inside of the body or the outside of the body without erroneous detection based on whether the infrared rays which are matched with this blinking pattern have been detected.

It is to be noted that the determination section 16 is connected with a non-illustrated light source control section of the light source apparatus 28 and can output a determination result to the light source control section. As a result, the light source control section can set a light volume upper limit based on MPE for eyes in case of the outside of the body, assume a light volume upper limit based on MPE for skin in case of the inside of the body, and control the excitation light source 30 so that a light volume required for observation can be obtained.

### Second Embodiment

A second embodiment according to the present invention will now be described.

It is to be noted that this second embodiment will be also explained as an endoscopic system using a biological endoscope. A portion different from the first embodiment alone will be described below.

In the first embodiment, the electromagnetic radiation unit 12 is previously installed at such as position at the trolley T or the main body section 20 mounted thereon as that the opening portion of the lumen, for example, the mouth of the subject can be irradiated with certainty with the infrared rays. On the other hand, in this second embodiment, the electromagnetic radiation unit 12 is configured as an independent unit that can be attachable to or detachable from an arbitrary position.

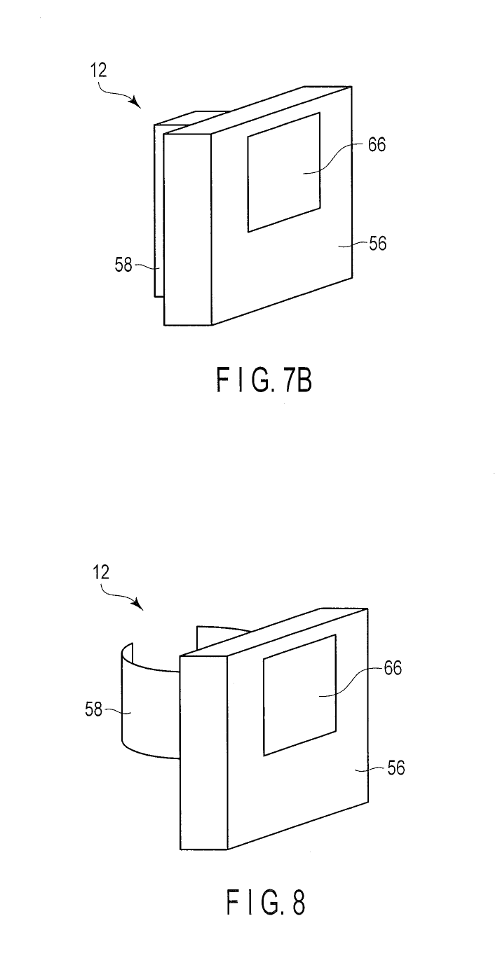

For example, as shown in FIG. 7A and FIG. 7B, the infrared LED 50, an electromagnetic wave control circuit 52, and others are mounted in one housing 56, and an attachment member 58 which is configured to fix the housing 56 at an arbitrary position is provided on the housing 56. Here, the attachment member 58 is configured as a hook that is hanged and put on an attachment portion such as a pocket of an operator (a physician or a nurse). Further, the attachment member 58 has a cleat 60 so that an attached state on the attachment portion can be maintained with certainty. In the housing 56, the infrared LED 50 and the electromagnetic wave control circuit 52 are assembled on a common wiring board 62, and a battery 64 that supplies electric power to these members through a non-illustrated wiring line on the wiring substrate 62 is further mounted. Furthermore, in the housing 56, an infrared ray radiation window 66 through which infrared rays pass is provided at a position corresponding to the infrared LED 50.

Moreover, as shown in FIG. 8, the attachment member 58 may be configured as a hook which is of a type that is disposed to hold a frame of a bed on which a subject lies or an arm of an operator.

In this manner, the attachment member 58 having a shape or a function suitable for the attachment portion can be selected and provided to the housing 56.

At the time of inserting an insertion portion 10 into an insertion opening I of a lumen of a subject, the electromagnetic radiation unit 12 having the above-described configuration can be disposed at a position that enables irradiating with greater certainty the insertion opening I of the lumen with infrared rays. For example, the electromagnetic radiation unit 12 can be disposed on an operator's chest or arm or a pole or a frame of a bed at a position enabling directly or indirectly illuminating the insertion portion 10. Besides, the electromagnetic radiation unit 12 can be disposed on various places, for example, trolley T of the endoscopic system or a wall in an examination room.

According to the configuration of this embodiment, since the electromagnetic radiation unit 12 can be moved to an optimum position, the electromagnetic radiation unit 12 can be arranged at an optimum position in accordance with, for example, a positional relationship of an operator, a subject, and a scope section 18 which varies depending on a type of examination and the like. That is, the electromagnetic radiation unit 12 can be arranged at an optimum position in accordance with an environment in a hospital.

Furthermore, since the electromagnetic radiation unit 12 uses the battery 64 and wiring lines and others are not provided, a freedom degree for arrangement is high.

### Third Embodiment

A third embodiment according to the present invention will now be described.

It is to be noted that this third embodiment will be also explained as an endoscopic system using a biological endoscope. A portion different from the first embodiment alone will be described hereinafter.

In the first embodiment, the electromagnetic radiation unit 12 is previously installed at such as position in the trolley T or the main body section 20 mounted therein as that the insertion opening I of the lumen, for example, the mouth of the subject can be irradiated with certainty with the infrared rays. On the other hand, in this third embodiment, an electromagnetic radiation unit 12 is mounted at a scope section 18 together with an electromagnetic wave detector 14.

For example, the electromagnetic radiation unit 12 is mounted at an operating portion 38 (see FIG. 3) of a scope section 18. In more detail, the electromagnetic radiation unit 12 is mounted at a portion which is near a connecting portion at which the operating portion 38 is connected to a bending portion 40 of the insertion portion 10 and is an end of an operating portion 38 (a hard member made of plastic or the like which does not readily deform) side. In particular, an infrared LED 50 (an infrared ray radiation window) of the electromagnetic radiation unit 12 is arranged at a region of the operating portion 38 that is touched by an operator's hand. Specifically, the infrared LED 50 is disposed on a scope distal end portion side of the operating portion 38 at a position that faces a right-hand side when the operator holds the operating portion 38 with his/her left hand.

That is, the operator holds a grip portion 68 of the operating portion 38 with his/her left and, holds a position near the distal end of the insertion portion 10 with his/her right hand, and carries the insertion portion 10 to an insertion opening I of a lumen of a subject irrespective of the operator's dominant hand. Therefore, in this embodiment, when the operator grips the grip portion 68 of the operating portion 38 with his/her left hand, the electromagnetic radiation unit 12 is mounted at a position on which the left hand is not placed and also a position facing the right hand side. As a result, the electromagnetic radiation unit 12 can radiate with greater certainty infrared rays toward the distal end of the insertion portion 10 held with the right hand.

As a result, the infrared rays can be prevented from being blocked by the operator, the subject, and other members in the examination room or the like, certainty for detecting in which one of the inside of a body and the outside of a body the insertion portion 10 is present can be improved.

It is to be noted that as shown in FIG. 10, the electromagnetic radiation unit 12 may be provided at a position on the main body section 20 side rather than the grip portion 68 of the operating portion 38. In this case, likewise, it is desirable for the electromagnetic radiation unit 12 to face the right side when an operator holds the operating portion 38 with his/her left hand.

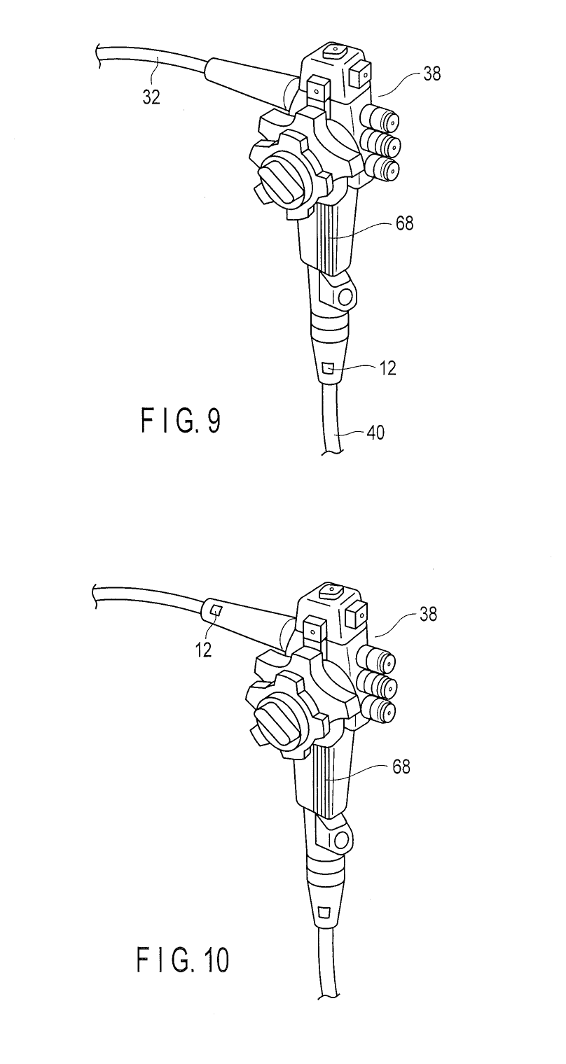

Since such a position as shown in FIG. 10 rather than such a position as shown in FIG. 9 enables arranging the electromagnetic radiation unit 12 near the center of gravity of the grip portion 68, an operator hardly feels a size and a weight of the electromagnetic radiation unit 12. Therefore, without a feeling of strangeness, it is possible to acquire an effect that infrared rays can be prevented from being blocked by the operator, the subject, or any other member in the examination room and certainty of detecting in which one of the inside or the outside of the subject's body the insertion portion 10 is present can be improved.

In addition, it is needless to say that the position of the electromagnetic radiation unit 12 may be any other position in the scope section 18 as long as it is a position that is never grabbed by an operator and also a position facing the opening portion of the lumen, for example, the subject's mouth.

### Fourth Embodiment

A fourth embodiment according to the present invention will now be described. It is to be noted that this fourth embodiment will be also described as an endoscopic system using a biological endoscope.

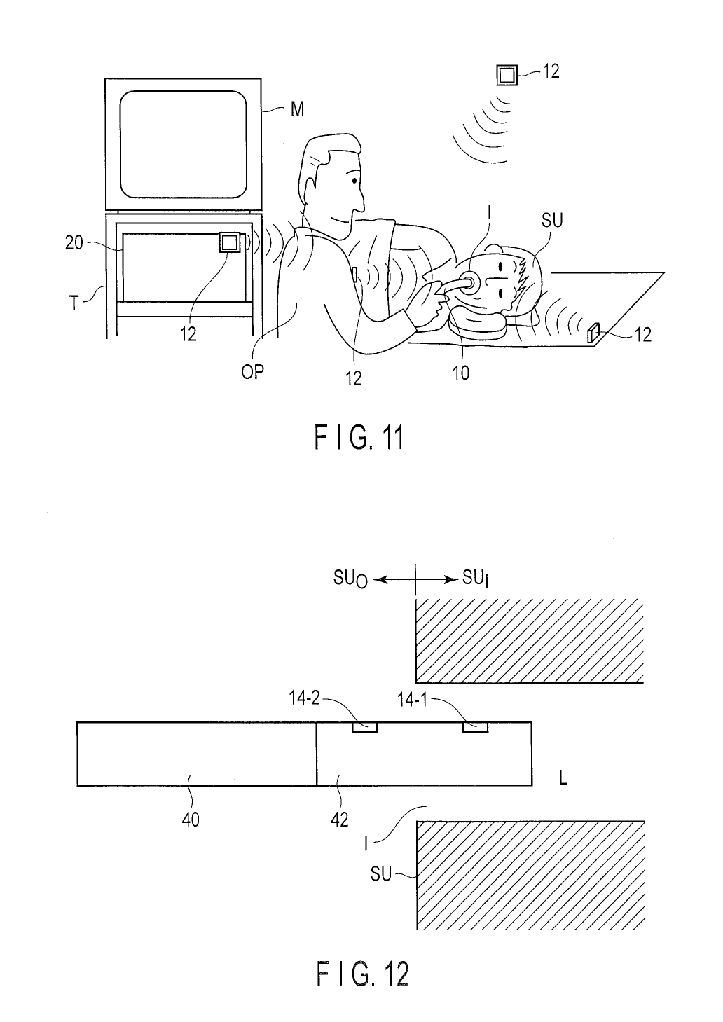

Although the single electromagnetic radiation unit 12 alone is provided in each of the first to third embodiments, the electromagnetic radiation units 12 are arranged to eliminate blind spots in this embodiment as shown in FIG. 11.

That is, the electromagnetic radiation units 12 according to the first to third embodiments are combined and they are disposed at various positions. For example, they are disposed on a trolley T, a main body section 20, a pocket or an arm of an operator (a physician or a nurse) OP, a subject SU himself/herself, a frame of a bed on which the subject SU lies, a wall in an examination room, a scope section 18, and others.

It is to be noted that, in this case, infrared rays radiated from the electromagnetic radiation units 12 all must have the same wavelength and predetermined blinking patterns must be synchronized. To achieve synchronization, for example, one electromagnetic radiation unit 12 can be determined as a base unit, and other units can be synchronized with this base unit. Since the fast and precise synchronization at the level of, for example, microseconds is not required, and hence the synchronization is easy.

As described above, in this embodiment, by enabling emission of the same infrared rays from the points, blind spots are eliminated, thereby improving certainty of detection.

### Fifth Embodiment

A fifth embodiment according to the present invention will now be described. It is to be noted that, likewise, this fifth embodiment will be described as an endoscopic system using a biological endoscope.

In each of the first to fourth embodiments, one electromagnetic wave detector 14 is arranged at the hard portion 42 at the distal end of the insertion portion 10 in the scope section 18. On the other hand, in this embodiment, the electromagnetic wave detectors 14 are arranged at the hard portion 42, and obtaining a difference between detection results from these detectors enables detecting with certainty that the hard portion 42 at the distal end of the insertion portion 10 in the scope section 18 is present in the inside of a body or the outside of a body.

That is, as shown in FIG. 12, a first electromagnetic wave detector 14-1 and a second electromagnetic wave detector 14-2 are mounted at the hard portion 42 at a predetermined interval. Here, assuming that the first electromagnetic wave detector 14-1 is provided on the distal end side and the second electromagnetic wave detector 14-2 is provided on the side near a hand, if the entire hard portion 42 is present in the outside of a body SU_{O} of a subject SU, the first and second electromagnetic wave detectors 14-1 and 14-2 detect substantially the same infrared rays. At the time of inserting the hard portion 42 into a lumen L (the inside of the body) of the subject SU, the first electromagnetic wave detector 14-1 first no longer detects infrared rays. At this time, the second electromagnetic wave detector 14-2 keeps detecting electromagnetic waves. Furthermore, when the hard portion 42 is completely inserted in the inside of the body SU_{I} of the subject SU, both the first and second electromagnetic wave detectors 14-1 and 14-2 abandon detecting infrared rays.

Therefore, when the first and second electromagnetic wave detectors 14-1 and 14-2 are normally operating, the following three situations can be considered.

(1) Both the detectors detect substantially the same infrared rays. In this case, it can be determined that the hard portion 42 at the distal end of the insertion portion 10 is completely present in the outside of the body SU_{O}.

(2) A detection amount (a light volume of infrared rays) of the first electromagnetic wave detector 14-1 is smaller than that of the second electromagnetic wave detector 14-2, and it gradually slightly varied. In this case, it can be determined that the hard portion 42 at the distal end of the insertion portion 10 is gradually inserted into the lumen L (the inside of the body SU_{I}).

(3) Neither detector detects the infrared rays. In this case, the hard portion 42 at the distal end of the insertion portion 10 can be determined to be completely present in the lumen L.

It is to be noted that, in case of other than the above-described three patterns, the electromagnetic waves from the electromagnetic radiation unit 12 are not correctly radiated, or an obstacle or the like has an impact. That is, in such a case, it may not be possible to correctly detect that the hard portion 42 at the distal end of the insertion portion 10 is present inside or outside the lumen L (the inside of the body SU_{I} or the outside of the body SU_{O}).

As described above, in this embodiment, the two electromagnetic wave detectors 14-1 and 14-2 are used, and detection amounts of the two detectors are compared, thereby detecting with greater certainty in which one of the inside of the body SU_{I} and the outside of the body SU_{O} of the subject the insertion portion 10 is present.

Further, whether the mechanism that detects the inside or the outside of the lumen is normally operating can be confirmed.

### Sixth Embodiment

A sixth embodiment according to the present invention will now be described. It is to be noted that this sixth embodiment will be likewise explained as an endoscopic system using a biological endoscope.

In each of the first to fourth embodiments, one electromagnetic wave detector 14 is arranged at the hard portion 42 at the distal end of the insertion portion 10 in the scope section 18. On the other hand, in this embodiment, a second electromagnetic wave detector is arranged at a position other than the hard portion 42, and obtaining a difference between detection results of these two detectors enables detecting with certainty that the hard portion 42 at the distal end of the insertion portion in the scope section 18 is present in the inside or the outside of a body.

That is, although the first electromagnetic wave detector 14-1 and the second electromagnetic wave detector 14-2 are mounted at the hard portion 42 in the fifth embodiment, a second electromagnetic wave detector 14-2 is disposed to, for example, an operating portion 38 in a scope section 18 in this embodiment. When such a configuration is adopted, it is possible to confirm from a detection result (an amount of receiving infrared rays) of the second electromagnetic wave detector 14-2 that an electromagnetic radiation unit 12 is normally operating. Furthermore, since it is possible to recognize intensity of electromagnetic waves (the infrared rays) to confirm whether the electromagnetic waves have reached the scope section 18, comparing this intensity with detection intensity of the first electromagnetic wave detector 14-1 provided at the hard portion 42 at the distal end of the insertion portion 10 enables improving the certainty of detection.

Moreover, the second electromagnetic wave detector 14-2 may be disposed to, for example, a main body section 20. When such a configuration is adopted, it is possible to confirm from a detection result (an amount of receiving infrared rays) of the second electromagnetic wave detector 14-2 that the electromagnetic radiation unit 12 is normally operating.

### Seventh Embodiment

A seventh embodiment according to the present invention will now be described.

In each of the first to sixth embodiments, the electromagnetic radiation unit 12 is arranged in the outside object O_{O}, and the electromagnetic wave detector 14 (14-1 and 14-2, or 14-1) is arranged at the insertion portion 10. However, an arrangement relationship between the electromagnetic radiation unit 12 and the electromagnetic wave detector 14 may be inverted.

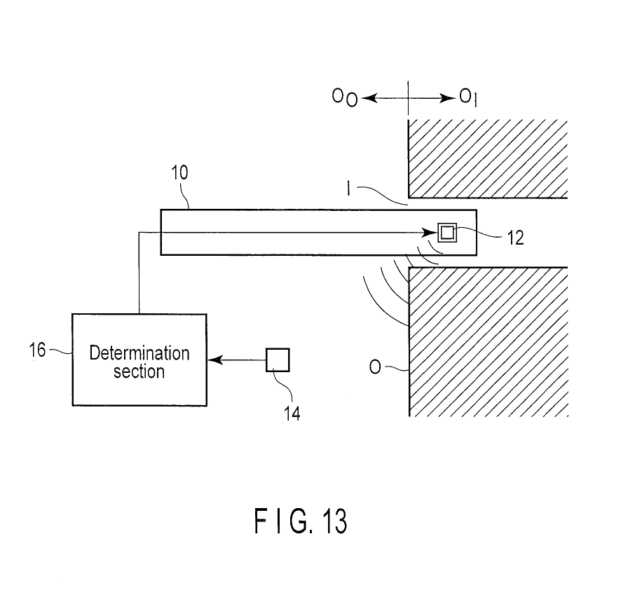

That is, as shown in FIG. 13, like the first to sixth embodiments, an endoscopic system according to this seventh embodiment is constituted of an insertion portion 10 of an endoscope that is inserted from an insertion opening I of an object O, an electromagnetic radiation unit 12 that radiates electromagnetic waves, an electromagnetic wave detector 14 as a detection section that detects the electromagnetic waves radiated from the electromagnetic radiation unit 12, and a determination section 16 that determines whether the insertion portion 10 is present in the inside of object O_{I} based on a detection result of the electromagnetic wave detector 14. However, in this seventh embodiment, the electromagnetic radiation unit 12 is arranged at the insertion portion 10, and the electromagnetic wave detector 14 is arranged at the outside of object O_{O}.

Even such a configuration can obtain the same effects as those of the first to sixth embodiments.

Although the present invention has been described based on the embodiments, the present invention is not restricted to the foregoing embodiments, and it can be modified or applied in many ways within the gist of the present invention as a matter of course.

For example, in each of the first to seventh embodiments, the example of the biological endoscope has been described, but the present invention can be likewise applied to an industrial endoscope. In this case, the determination section 16 can be connected to a non-illustrated light source control section of a light source apparatus 28, and it can output its determination result to the light source control section. As a result, the light source control section can dim or stop illumination light in order to extend life duration of the light source apparatus 28 or achieve power saving in case of the outside of object O_{O}, or it can control an excitation light source 30 so that a light volume required for observation can be obtained in case of the inside of object O_{I}.

Additionally, the light source apparatus 28 may use a scattering section that performs scattering without converting a wavelength, or an exiting light characteristic converting section that converts a spread angle of a beam, in place of the wavelength converting section 34.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. An endoscopic system which an insertion portion of an endoscope is inserted from an insertion opening of an object to observe an inner surface of the object, comprising:
an electromagnetic radiation unit configured to radiate electromagnetic waves;
a detection section configured to detect the electromagnetic waves; and
a determination section configured to determine whether the insertion portion is present in the object based on a detection result of the detection section,
wherein one of the electromagnetic radiation unit and the detection section is arranged outside the object, and the other is arranged at the insertion portion.

2. The system according to claim 1, wherein
the endoscopic system is an endoscopic system which is used by inserting the insertion portion into a lumen of a living body,
the electromagnetic radiation unit is arranged outside the lumen, and
the detection section is arranged at the insertion portion.

3. The endoscopic system according to claim 2, wherein the electromagnetic radiation unit is one of an infrared radiation element configured to radiate infrared rays and an electromagnetic radiation element configured to radiate electric waves having a wavelength longer than that of the infrared rays.

4. The system according to claim 3, wherein
the endoscopic system is dividable into a scope section having the insertion portion and a main body section configured to process and display an observation image observed through the scope section,
the determination section is configured in the main body section,
the detection section is incorporated in the insertion portion of the scope section, and
the electromagnetic radiation unit is not provided for the scope section.

5. The system according to claim 4, wherein
the main body section is mounted at a trolley, and
the electromagnetic radiation unit is previously incorporated in at least one of the main body section and the trolley.

6. The system according to claim 4, wherein the electromagnetic radiation unit is configured to be attachable to and detachable from an arbitrary position.

7. The system according to claim 3, wherein
the endoscopic system is dividable into a scope section having the insertion portion and a main body section configured to process and display an observation image observed through the scope section,
the determination section is configured in the main body section,
the electromagnetic radiation unit is arranged at a region excluding the insertion portion of the scope section, and
the detection section is arranged at the insertion portion of the scope section.

8. The system according to claim 7, wherein the electromagnetic radiation unit is mounted at an operating portion of the scope section.

9. The system according to claim 7, wherein
at least a distal end portion of the insertion portion of the scope section is formed as a hard portion, and
the detection section is arranged at the hard portion of the insertion portion.

10. The system according to claim 2, comprising a plurality of the electromagnetic radiation units.

11. The system according to claim 2, further comprising a second detection section configured to detect the electromagnetic waves,
wherein the determination section is configured to determine whether the insertion portion is present in the object based on a difference between a detection result of the detection section and a detection result of the second detection section.

12. The system according to claim 2, wherein the electromagnetic radiation unit configured to radiate electromagnetic waves modulated in a predetermined pattern as the electromagnetic waves.
